(19) United States
(12) Patent Application Publication
(10) Pub. No.: US 2013/0109842 A1
(43) Pub. Date: May 2, 2013

(54) AMINO ACID SEQUENCES OF NANOBODIES DIRECTED AGAINST P19 SUBUNIT OF THE HETERODIMERIC CYTOKINE IL-23

(71) Applicants: Veronique De Brabandere, Gent (BE); Marc Jozef Lauwereys, Haaltert (BE); Erika Morizzo, Gent (BE); Heidi Rommelaere, Gent (BE); Ann Union, Aalter (BE); Gert Verheyden, Bavegem (BE)

(72) Inventors: Veronique De Brabandere, Gent (BE); Marc Jozef Lauwereys, Haaltert (BE); Erika Morizzo, Gent (BE); Heidi Rommelaere, Gent (BE); Ann Union, Aalter (BE); Gert Verheyden, Bavegem (BE)

(73) Assignee: Ablynx N.V., Zwijnaarde (BE)

(21) Appl. No.: 13/695,312

(22) PCT Filed: Apr. 28, 2011
(86) PCT No.: PCT/EP2011/056736

§ 371 Date: Dec. 28, 2012

Related U.S. Application Data
(60) Provisional application No. 61/329,908, filed on Apr. 30, 2010.
(60) Provisional application No. 61/348,799, filed on May 27, 2010.

(51) Int. Cl. C07K 16/24; C07K 14/76; C07K 16/46

(52) U.S. Cl. CPC C07K 16/244; C07K 16/46; C07K 16/468; C07K 14/76
USPC 530/387.3

(57) ABSTRACT

The present invention relates to amino acid sequences that are directed against the p19 subunit of IL-23; as well as protein, constructs and compounds comprising the same; and also nucleic acids encoding the same.

The present invention relates to amino acid sequences that are directed against the p19 subunit of IL-23; as well as protein, constructs and compounds comprising the same; and also nucleic acids encoding the same.

Further aspects, embodiments, features, advantages, uses, applications and advantages from the present invention will become clear from the further description herein.

The international application WO 09/068,627 by applicant entitled “Amino acid sequences directed against heterodimeric cytokines and/or their receptors and polypeptides comprising the same” describes amino acid sequences against a subunit of the heterodimeric cytokine IL-23 called p19 (also called IL-30B, Genbank accession number NM_{—}016584. Reference is also made to the prior art on heterodimeric cytokines cited in WO 09/068,627, for example in the paragraph bridging pages 2 and 3).

Unless explicitly mentioned otherwise herein, all terms mentioned herein have the meaning given in WO 09/068,627 (or in the prior art cited in WO 09/068,627). Also, where a method or technique is not specifically described herein, it can be performed as described in WO 09/068,627 (or in the prior art cited in WO 09/068,627). For example, the term “Nanobody” is as defined in WO 09/068,627, and thus in a specific aspect generally denotes a VHH, a humanized VHH or a camelized VH (such as a camelized human VH).

WO 09/068,627 describe a number of amino acid sequences that are directed against (as defined in WO 09/068,627) p19 (see for example the “P19+” and “P19−” sequences mentioned in Table A-2 of WO 09/068,627, as well as FIGS. 20-21 and the examples of WO 09/068,627). WO 09/068,627 also describes humanized variants of these P19 sequences, as well as multivalent, multispecific and/or biparatopic constructs (as defined in WO 09/068,627) that are directed against (the p19 subunit present in) IL-23 and that comprise at least one such p19 sequence. Reference is for example made to the constructs referred to on pages 173 (last paragraph) to 175 (first paragraph) of WO 09/068,627, as well as FIGS. 30, 31, 32 and 33 of WO 09/068,627 and the examples of WO 09/068,627).

One particularly preferred example of an amino acid sequence against the P19 subunit from WO 09/068,627 is the sequence called 119A3 (see SEQ ID NO: 1898 in WO 09/068,627):

(SEQ ID NO: 1)
EVQLVESGGGLVQAGGSLRLSCAASGRIFSLPASGNIFNLLTIAWHRQAP
GMQRELVATINSGSRTNYADSVKGRFTISRDNAQKTVYLQMNNLKPEDTA
VYYCQTSGSGSPNFWGQGTQVTVSS

WO 09/068,627 also describes a number of variants of 119A3, and in particular the variants P23IL119A3(H37Y) (SEQ ID NO: 2559 in WO 09/068,627), P23IL119A3(M43K) (SEQ ID NO: 2560 in WO 09/068,627), P23IL119A3(H37Y-M43K) (SEQ ID NO: 2560 in WO 09/068,627, also given in the attached sequence listing as SEQ ID NO:2) and a series of humanized variants of 119A3 (with the H37Y and M43K mutations) called P23IL 119A3-BASIC and P23IL119A3V1 to P23IL119A3V17 (SEQ ID NOs: 2561 to 2579 in WO 09/068,627. The sequence of P23IL119A3V17 is also given in the attached sequence listing as SEQ ID NO:3).

WO 09/068,627 further gives some non-limiting examples of multivalent, multispecific and/or biparatopic constructs that comprising 119A3 or humanized variants thereof (see for example SEQ ID NO's: 2151 to 2159, 2533, 2537, 2539, 2541 to 2547 and 2615 to 2628).

The U.S. provisional application 61/181,384 filed by Ablynx N. V. on May 27, 2009 and entitled “Biparatopic protein constructs directed against IL-23” describes biparatopic protein constructs directed against IL-23, including constructs that comprise 119A3 and humanized variants thereof. This application in Example 1 also describes the binding interactions of 119A3 and the p19 subunit of IL-23.

Generally, the anti p19 sequences and constructs from WO 09/068,627 and the U.S. provisional application 61/181,384 show excellent biological activity and other desired properties. However, this does not mean that an anti p19 sequence that would have (even further) improved properties would not be a valuable addition to the art.

The invention provides such improved p19 binders, and in particular (even further) improved variants of the sequences 119A3 (SEQ ID NO:1), P23IL119A3(H37Y-M43K) (SEQ ID NO:2) and PMP119A3v17 (SEQ ID NO:3).

The amino acid sequences provided by the invention are humanized variants of 119A3 that comprise, at position 78 (numbering according to WO 09/068,627), a valine residue. In a preferred aspect, an amino acid sequence of the invention may be a humanized variant of P23IL119A3 (H37Y-M43K) (SEQ ID NO:2) that comprises, at position 78 (numbering according to WO 09/068,627), a valine residue. In this respect, it should be noted that although the “wild type” PMP119A3 naturally contains a V at position 78, and although WO 09/068,627 also describes three variants of PMP119A3—called P23IL119A3(H37Y) (see SEQ ID NO: 2559) P23IL119A3(M43K) (see SEQ ID NO: 2560); and P23IL119A3(H37Y-M43K) (see SEQ ID NO: 2561), respectively—that contain a V at position 78, that neither of these variants contain any humanizing substitutions as defined herein (as described below, the mutation H37Y is a mutation at a “Hallmark residue”, and the mutation M43K replaces one “camelid residue” with another “camelid residue”). It should also be noted that all the humanized variants of 119A3 described in WO 09/068,627 (called “P23IL119A3Basic” through to “P23IL119A3v17” in WO 09/068,627) contain, in addition to the two mutations H37Y and M43K and at least one humanizing substitution (such as A14P, Q75K or N82bS), a leucine at position 78.

Generally, a “humanized variant” of an amino acid sequence is a variant that comprises one or more “humanizing” substitutions, as defined herein. Preferably, compared to the wild-type sequence 119A3, an amino acid sequence of the invention contains at least one such humanizing substitution, and preferably at least two such humanizing substitutions, and preferably at least three humanizing substitutions. Also, again compared to the wild-type sequence 119A3, the amino acid sequences of the invention preferably comprise a maximum of seven humanizing substitutions, and preferably a total of three, four or five humanizing substitutions (although the maximum number may in some cases not be critical, depending on the humanizing substitutions chosen). Some preferred, but non-limiting examples of such humanizing substitutions will become clear from the further description herein, and for example include, without limitation, A14P, Q75K, N82bS and/or A49S.

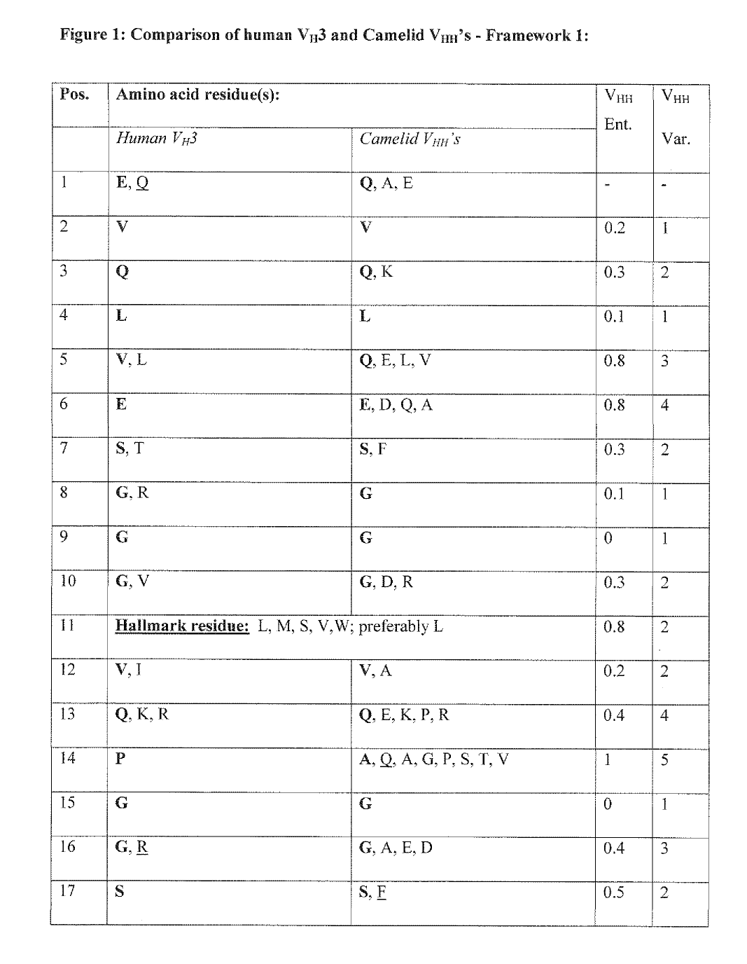

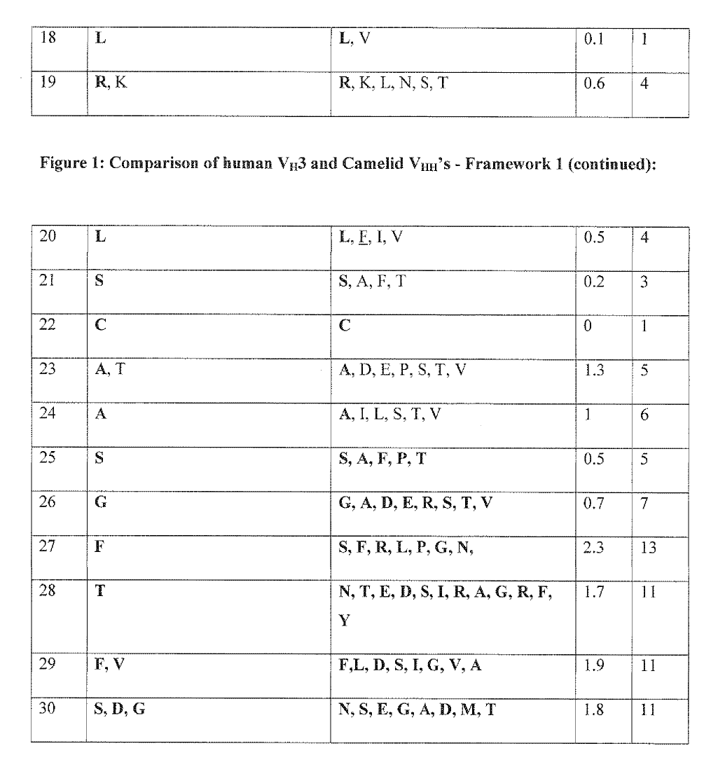

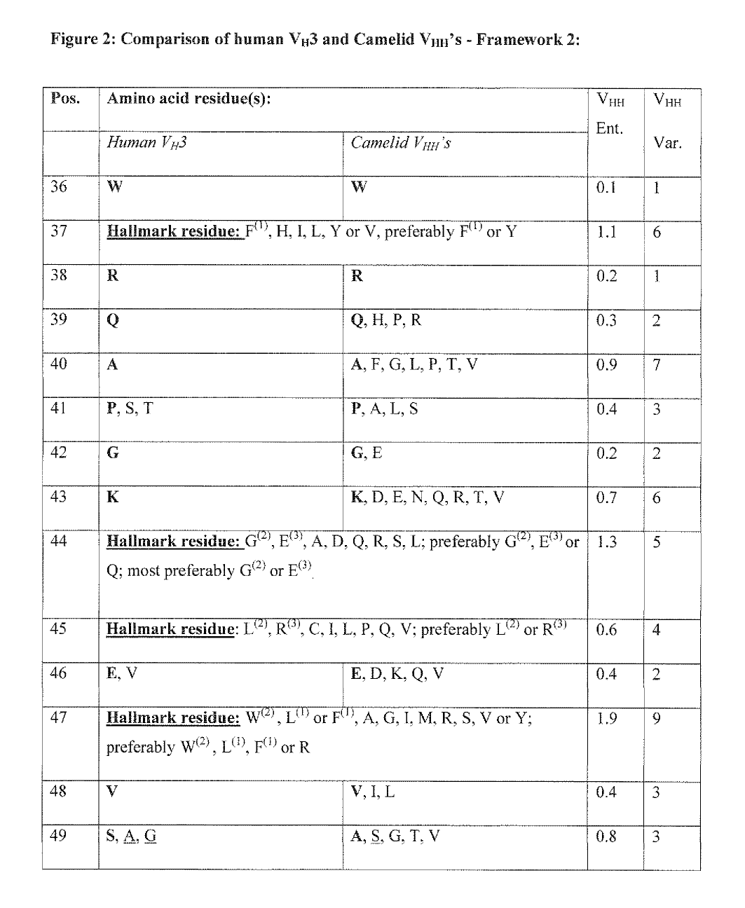

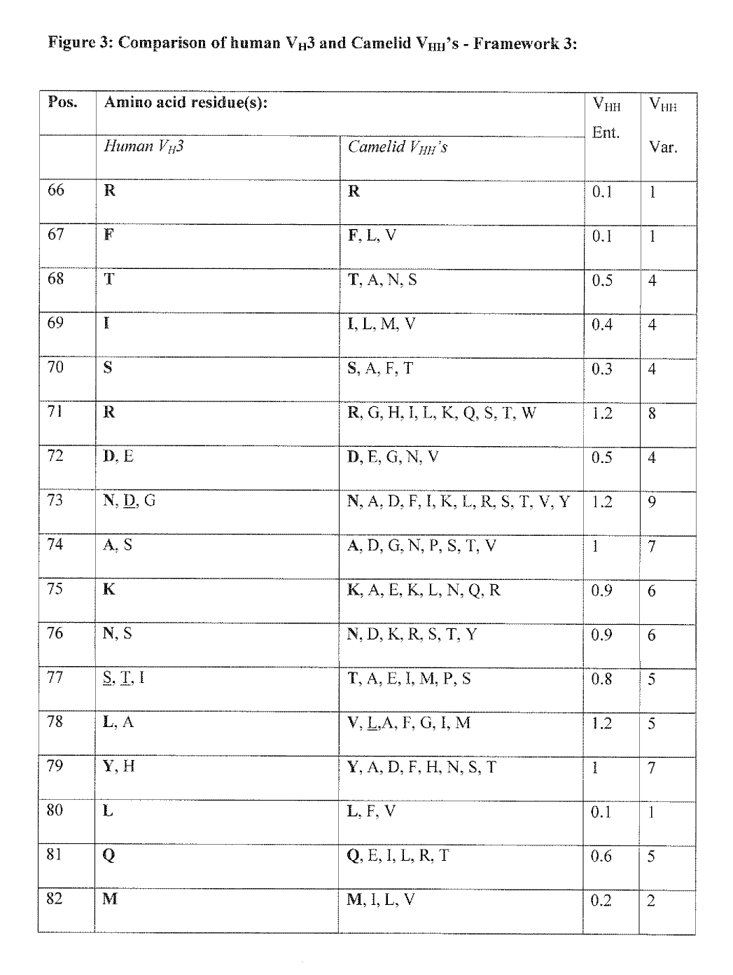

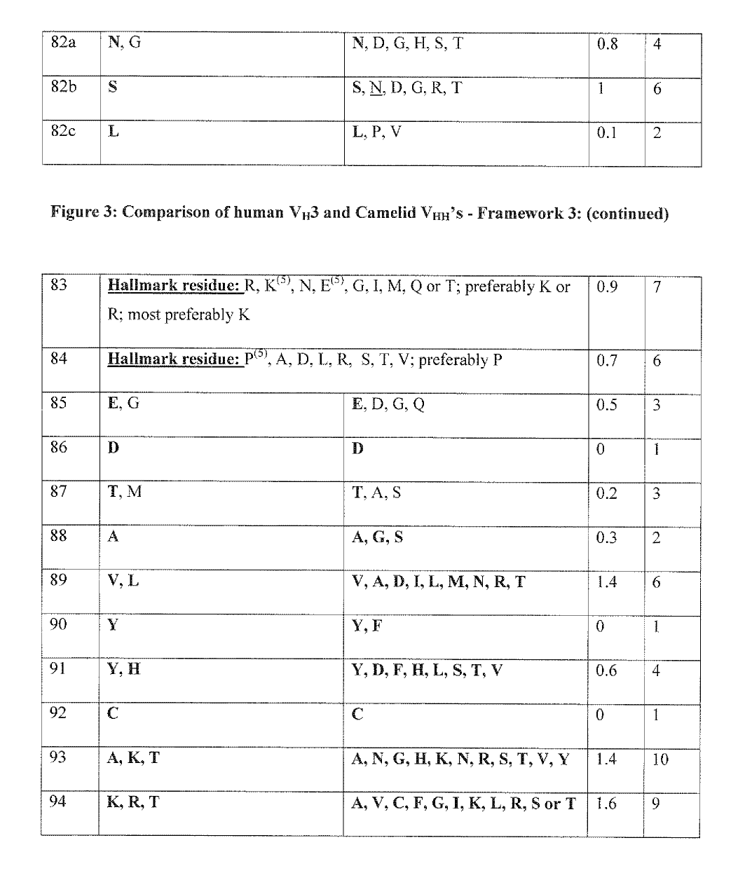

Also, as further described herein, the amino acid sequences of the invention may contain one or more other/further substitutions. Again, some preferred, but non-limiting examples of such other/further substitutions will become clear from the further description herein, and for example may include (and preferably essentially consist of) one or more of the following substitutions (also referred to herein as “substitutions (a) to (c)”):
(a) one or more conservative amino acid substitutions; and/or
(b) one or more substitutions in which a “camelid” amino acid residue at a certain position is replaced by a different camelid” amino acid residue that occurs at said position (for which reference is for example made to FIGS. 1-4). Some non-limiting examples of such substitutions are V5L, M43K (substitution to the residue that is most prevalent in this position in both human VH's as well as VHH's). S49A and/or A74S; and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific example of suitable substitutions (see for example pages 4 and 5 and pages 10 to 15). One example of such substitution may be to replace an NS motif at positions 82a and 82b with an NN motif;
   or any suitable combination of two or more of any of the foregoing substitutions (a) to (c).

It will be clear from the disclosure herein that the amino acid sequences of the invention contain at least one “amino acid difference” compared to each of the sequences of 119A3, P23IL119A3(H37Y-M43K) and 119A3v17, respectively (in which the term “amino acid difference” is used herein in the same meaning as defined in WO 09/068,627, namely as an insertion, deletion or substitution of a single amino acid residue on a position of the first sequence, compared to the second sequence; it being understood that two amino acid sequences can contain one, two or more such amino acid differences. In the context of the present invention, any amino acid difference is preferably a substitution).

In particular, compared to the sequences of 119A3 and P23IL119A3(H37Y-M43K), the amino acid sequences of the invention contain at least one humanizing substitution (as defined herein), and may optionally contain one or more further substitutions (such as any one of, or any suitable combination of any two or more of, the further substitutions (a) to (c) as mentioned herein).

Compared to the humanized sequence 119A3v17, the amino acid sequences of the invention contains at least the substitution L78V, and may optionally one or more further humanizing substitutions (as described herein) and/or may optionally one or more further substitutions (such as any one of, or any suitable combination of any two or more of, the further substitutions (a) to (c) as mentioned herein); or a suitable combination of such humanizing substitutions and such other substitutions.

In a preferred aspect, the amino acid sequences of the invention contain a total of between 7 and 15, preferably between 9 and 13, such as 10, 11 or 12 amino acid differences (as defined herein by reference to the definition used in WO 09/068,627) compared to the wild-type sequence of 119A3. As mentioned, these differences preferably at least comprise one and preferably both of the substitutions H37Y and/or M43K, and at least one, preferably at least two, such as three, four or five humanizing substitutions, and may optionally one or more further substitutions (such as any one of, or any suitable combination of any two or more of, the further substitutions (a) to (c) as mentioned herein). Again, based on the disclosure herein and optionally after a limited degree of trial and error, the skilled person will be able to select (a suitable combination of) one or more such suitable humanizing and/or further substitutions.

In another specific aspect, the amino acid sequences of the invention contain a total of between 1 and 5, such as one, two or three amino acid differences compared to the sequence of 119A3v17, in which at least one of these amino acid differences is the substitution L78V and the other substitutions may for example be, and preferably are, either one or more further humanizing substitutions (compared to the humanizing substitutions already present in 119A3v17) and/or one or more further substitutions (such as any one of, or any suitable combination of any two or more of, the further substitutions (a) to (c) as mentioned herein). Again, based on the disclosure herein and optionally after a limited degree of trial and error, the skilled person will be able to select (a suitable combination of) one or more such suitable humanizing and/or further substitutions.

Also, most preferably, these amino acid differences compared to 119A3 and/or 119Av17 are most preferably located in the framework regions (defined according to Kabat, reference is again made to WO 09/068,627), although it is not fully excluded that a very limited number of these amino acid differences (such as for example only one or two) may be present in the CDR's (as long as these do not detract (too much) from the desired affinity, on-rate or off-rate (for example, such amino acid differences in the CDR's may be introduced as a result of affinity maturation). A preferred, but non-limiting example of a suitable substitution in one of the CDR's may be the substitution N52E.

A preferred, but non-limiting example of an amino acid sequence of the invention is the sequence 119A3v18, which corresponds to the sequence 119A3v17, but with a valine at position 78 (where 119A3v17 comprises a leucine at position 78).

(SEQ ID NO: 4)
EVQLLESGGGLVQPGGSLRLSCAASGRIFSLPASGNIFNLLTIAWYRQAP
GKGRELVATINSGSRTYYADSVKGRFTISRDNSKKTVYLQMNSLRPEDTA
VYYCQTSGSGSPNFWGQGTLVTVSS

Other amino acid sequences of the invention may for example contain a total of between one and five, such as one, two or three amino acid differences compared to the sequence of 119A3v18 (while retaining the valine at position 78), in which such amino acid differences may for example be, and most preferably are, either one or more further humanizing substitutions (such as, for example the humanizing substitution A49S) and/or any one of, or any suitable combination of any two or more of, the further substitutions (a) to (c) as mentioned herein (such as for example, the substitutions N52E and/or S82bN). Some preferred, but non-limiting examples of such variants of 119A3v18 are 119A3v20 (SEQ ID NO: 5), 119A3v21 (SEQ ID NO: 6) and 119A3v22 (SEQ ID NO: 7), and these specific variants (and polypeptides of the invention comprising the same, as further described herein) form further aspects of the invention.

In the invention, generally, it has been found that humanized variants of 119A3 that comprise a valine at position 78 (and which are preferably as further described herein) are more stable, may give higher expression or production yields and/or may have other advantages compared to (the corresponding) humanized variants of 119A3 that comprise a leucine at position 78. Without being limited to any specific explanation or hypothesis, it is believed that this may be due to the fact that (the framework sequences of) humanized variants of 119A3 that comprise a valine at position 78 allow the Nanobody to are better able to fold into the desired immunoglobulin domain structure and/or that humanized variants of 119A3 that comprise a valine at position 78, upon folding, to take on a more stable immunoglobulin domain structure.

Thus, in a specific, but non-limiting aspect, the invention relates to an amino acid sequence (i.e. an amino acid sequence of the invention) that is a (humanized) variant of PMP119A3 (SEQ ID NO:1) that comprises, compared to the amino acid sequence of PMP119A3, (i) at least one and preferably both of the mutations H37Y and M43K; (ii) a valine residue at position 78; (iii) at least one, preferably at least two, and more preferably three, four of five humanizing substitutions (as defined herein); (iv) as well as optionally one or more further suitable amino acid substitutions (preferably, any one of, or any suitable combination of any two or more of, the further substitutions (a) to (c) as mentioned herein).

As mentioned, such a variant of 119A3 preferably contains (i) a total of between 7 and 15, preferably between 9 and 13, such as 10, 11 or 12 amino acid differences compared to the wild-type sequence of 119A3; and (ii) a maximum of seven humanizing substitutions, and preferably a total of three, four or five humanizing substitutions (although the maximum number may in some cases not be critical, depending on the humanizing substitutions chosen).

In another aspect, the invention relates to an amino acid sequence (i.e. an amino acid sequence of the invention) that is a (humanized) variant of 119A3(H37Y-M43K) (SEQ ID NO:2) that comprises, compared to the amino acid sequence of PMP119A3(H37Y-M43K), (i) a valine residue at position 78; (ii) at least one, preferably at least two, and more preferably three, four of five humanizing substitutions (as defined herein); (iii) as well as optionally one or more further suitable amino acid substitutions (preferably, any one of, or any suitable combination of any two or more of, the further substitutions (a) to (c) as mentioned herein). Such a variant of 119A3(H37Y-M43K) preferably contains (i) a total of between 5 and 13, preferably between 7 and 11, such as 9, 10 or 11 amino acid differences compared to the sequence of 119A3 (H37Y-M43K); and (ii) a maximum of seven humanizing substitutions, and preferably a total of three, four or five humanizing substitutions (although the maximum number may in some cases not be critical, depending on the humanizing substitutions chosen).

In another aspect, the invention relates to an amino acid sequence (i.e. an amino acid sequence of the invention) that is a variant of 119A3v17 that comprises, compared to the amino acid sequence of 119A3v17, (i) a valine residue at position 78; (ii) optionally 1 to 5, such as one, two or three further amino acid differences compared to the sequence of 119A3v17, in which said amino acid differences are preferably substitutions and more preferably substitutions that are chosen from one or more further humanizing substitutions (compared to the humanizing substitutions already present in 119A3v17) and/or from any one of, or any suitable combination of any two or more of, the further substitutions (a) to (c) as mentioned herein.

Some preferred, but non-limiting examples of amino acid sequences of the invention (and in particular, some preferred, but non-limiting examples of such variants of 119A3v17) include, without limitation 119A3v18, 119A3v20, 119A3v21 and 119A3v22 (SEQ ID NO's: 4 to 7), and these form further preferred aspects of the invention.

The invention also relates to proteins and polypeptides that comprise or essentially consist of an amino acid sequence of the invention.

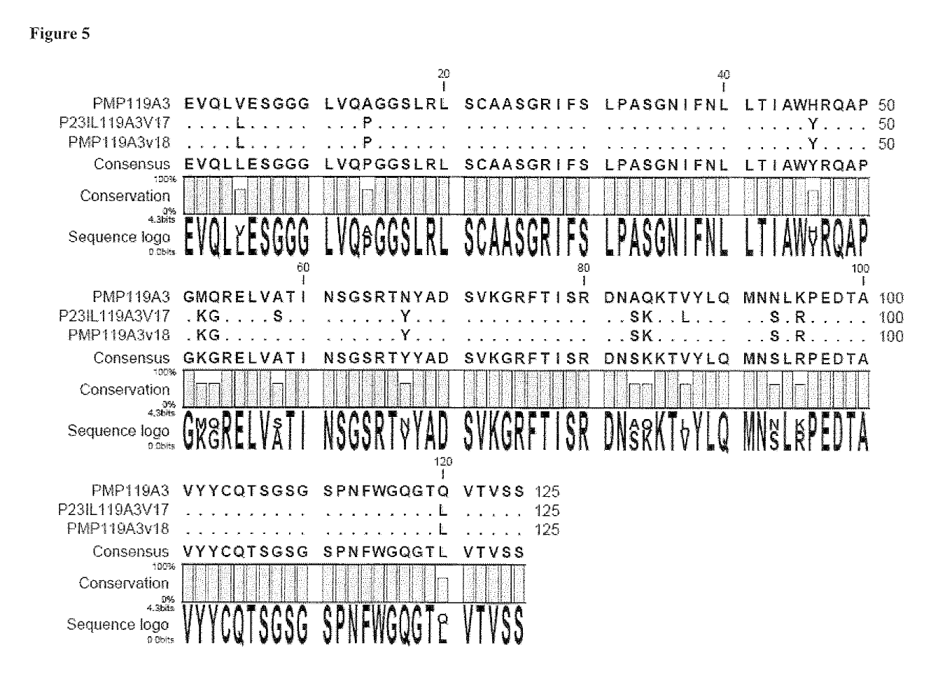

An alignment of 119A3 (WO 09/068,627), 119A3v16 (WO 09/068,627) and 119A3v18 (invention) is given in FIG. 5.

With respect to any humanizing substitutions that may be present in the amino acid sequences of the invention (i.e., compared to 119A3, 119A3 (H37Y-M43K) and/or 19A3v17), it is remarked that as described in WO 09/068,627, a humanizing substitution can generally be defined as a substitution whereby an amino acid residue that occurs in a framework regions of a camelid V_{HH} domain is replaced by a different amino acid that occurs at the same position in the framework region of a human V_{H} domain (and preferably, a human V_{H}3 domain). Thus, suitable humanizing substitutions will be clear to the skilled person based on the disclosure herein, the disclosure in WO 09/068,627, and from a comparison of the amino acid sequence of a given V_{HH} sequence and one or more human V_{H} sequences.

Reference is for example made to the attached FIGS. 1-4 (which have been taken from Tables A-6 to A-9 of WO 09/068,627), which list some of the amino acid residues that have been found to occur in the framework regions of camel id VHH domains, and the corresponding amino acid residue(s) that most often occur in the framework regions of a human V_{H}3 sequence (such as for example, the germline sequences DP-47, DP-51 or DP-29). The humanizing substitutions that can be taken from these Figures are also some of the preferred humanizing substitutions used in the invention; however, it may also be possible to use humanizing substitutions that have been obtained by comparison with other germline sequences (from the V_{H}3 class or sometimes also from other V_{H} classes). As generally known from WO 09/068,627 (and from the patent applications from Applicant and the further prior art mentioned in WO 09/068,627), based on such sequence comparison, particularly suited and/or optimal humanizing substitutions (and combinations thereof) may generally be determined by limited trial and error, i.e. by introducing one or more envisaged humanizing substitutions and testing the humanized variants thus obtained for one or more desired properties, such as melting temperature, affinity, potency, properties upon formatting, expression levels in a desired host organism, and/or other desired properties for VHH domains or Nanobodies or proteins/polypeptides comprising the same, for which again reference is made to WO 09/068,627 and the further patent applications by applicant mentioned therein).

For example, and without limitation, compared to the sequences of 119A3 and 119A3 (H37Y-M43K), an amino acid sequence of the invention may comprise one of, any two of and preferably all three of the substitutions A14P, Q75K and/or N82bS (although as mentioned, it may be desirable to have an NN motif at positions 82a/82b rather than a NS motif), which are all present in 119A3v17 (WO 09/068,627) and in 119A3v18 (invention); and may for example also comprise the substitution A49S (for example, compared to 119A3v18).

With regard to humanizing substitutions, it should be noted that for the purposes of the present application, any substitutions at any of the camelid “Hallmark residues” (see again WO09/068,627, as well as FIGS. 1-4) should not be counted as a “humanizing substitution”. Such substitutions at any of the Hallmark residues may or may not be present, and which when present may or may not be a substitution in which an amino acid residue in a VHH is replaced by an amino acid residue that occurs at the same position of a human V_{H} sequence. For example, such a substitution at a Hallmark residue may for example also be a substitution in which an amino acid residue that occurs at the Hallmark position is replaced by another amino acid residue that occurs at said position in camelid V_{HH} sequences (reference is again made to FIGS. 1-4).

Examples of such substitutions at Hallmark residues which may be present in the amino acid sequences of the invention, compared to the sequence of 119A3, are one of, any two, any three of and preferably all four of H37Y, Q44G, K84R and/or Q108L. These are all present in for example 119A3v17 (WO 09/068,627) and in 119A3v18 (invention).

Suitable examples of other/further substitutions that may be present, compared to for example the sequence of 119A3, 119A3v17 and/or 119A3v18, are any one of, or any suitable combination of any two or more of, the further substitutions (a) to (c) as mentioned herein, and/or for example also the substitution N52E.

It will be clear to the skilled person from the disclosure herein that the amino acid sequences of the invention are directed against IL-23 (and in particular, the p19 subunit of IL-23) and are improved variants for 119A3 and its (humanized) variants as described in WO 09/068,627. Thus, the amino acid sequences of the invention can be used for the same purposes, uses and applications as described in WO 09/068,627, for example to modulate signaling that is mediated by IL-23 and/or its receptor(s); and/or in the prevention or treatment of diseases associated with IL-23 and/or with signaling that is mediated by IL-23, such as for example inflammation and inflammatory disorders such as bowel diseases (colitis, Crohn'disease, IBD), infectious diseases, psoriasis, cancer, autoimmune diseases (such as MS), carcoidis, transplant rejection, cystic fibrosis, asthma, chronic obstructive pulmonary disease, rheumatoid arthritis, viral infection, common variable immunodeficiency, and the various diseases and disorders mentioned in the prior art cited herein. Further reference is made to WO 09/068,627.

In particular, the amino acid sequences of the invention are “p19+” sequences (as defined in WO 09/068,627), and thus may in particular be used for the same purposes as described in WO 09/068,627 for other “p19+ sequences”. Reference is for example made to pages 15 and 16 of WO 09/068,627, as well as the further general disclosure of WO 09/068,627. More in particular, the amino acid sequences of the invention may be used as an improved alternative to 119A3 and the humanized variants thereof, and thus may in particular be used for the same purposes as described in WO 09/068,627 for 119A3 and its humanized variants.

As already mentioned in WO 09/068,627, one of these applications of p19+ sequences and/or 119A3 and its humanized variants (and thus for the amino acid sequences of the invention) is as a building block in compounds or constructs that comprise, next to one or more amino acid sequences of the invention, and one or more other groups, residues, moieties, binding domains or binding units (as described in WO 09/068,627). For example, as described in WO 09/068,627, such one or more further binding domains or binding units may be other immunoglobulin single variable domains, VHH's, (single) domain antibodies, Nanobodies or dAb's, and these may for example be directed against p19 or against another subunit of a heterodimeric cytokine of the IL-12/IL-23 family, such as p40.

In particular, as described in WO 09/068,627, such a compound or construct may be a bivalent or multivalent, bispecific or multispecific, and/or biparatopic construct (as described in WO 09/068,627) that comprises, in addition to one or more (and preferably one) amino acid sequence of the invention, one or more other Nanobodies, such as, for example and without limitation, one or more of the Nanobodies described, mentioned and/or referred to in WO 09/068,627. Reference is for example made to pages 12 to 16, pages 27 to 30 and to the constructs comprising at least one p19+ sequence as mentioned on page 31 of WO 09/068,627. As mentioned therein, such another Nanobody may for example be another Nanobody against p19 or a Nanobody against p40, or any other suitable Nanobody. As also described in WO 09/068,627, see for example pages 172-173, such compounds or constructs may also have been modified to increase their half-life (e.g. by circulation) or may comprise and/or may be fused to a protein or binding unit that provides for increased half-life (for example, albumin or a binding unit or binding peptide that can bind to a serum protein such as albumin).

Such compounds and (bivalent or multivalent, bispecific or multispecific, and/or biparatopic) constructs are preferably proteins and polypeptides (i.e. encoded by a nucleotide sequence and/or capable of being expressed by a host or host cell), as also generally described in WO 09/068,627. They are also generally referred to herein as “polypeptides of the invention”. Some preferred, but non-limiting examples of such polypeptides of the invention are given in SEQ ID NO's 8 to 40, and these polypeptides form some preferred aspects of the invention.

Thus, the “polypeptides of the invention” as described herein may be essentially as described for the “polypeptides of the invention” described in WO 09/068,627, but comprising at least one amino acid sequence of the invention (i.e. instead of another P19+ sequence that may be present in a “polypeptides of the invention” as described in WO 09/068,627).

In particular, and preferably, a “polypeptides of the invention” as described herein may be essentially as described for the “polypeptides of the invention” described in WO 09/068,627 that comprise p119A3 or one of the variants thereof described in WO 09/068,627, but comprising an amino acid sequence of the invention where a “polypeptides of the invention” as described in WO 09/068,627 comprises 119A3 or one of the variants thereof described in WO 09/068,627. In other words, some preferred examples of polypeptides of the invention may be obtained simply by taking one of the polypeptides of the invention as described in WO 09/068,627 that comprises 119A3 or one of the variants thereof described in WO 09/068,627, and replacing 119A3 or said variant with an amino acid sequence of the invention (for example, and preferably, with 119A3v18, 119A3v20, 119A3v21 or 119A3v22).

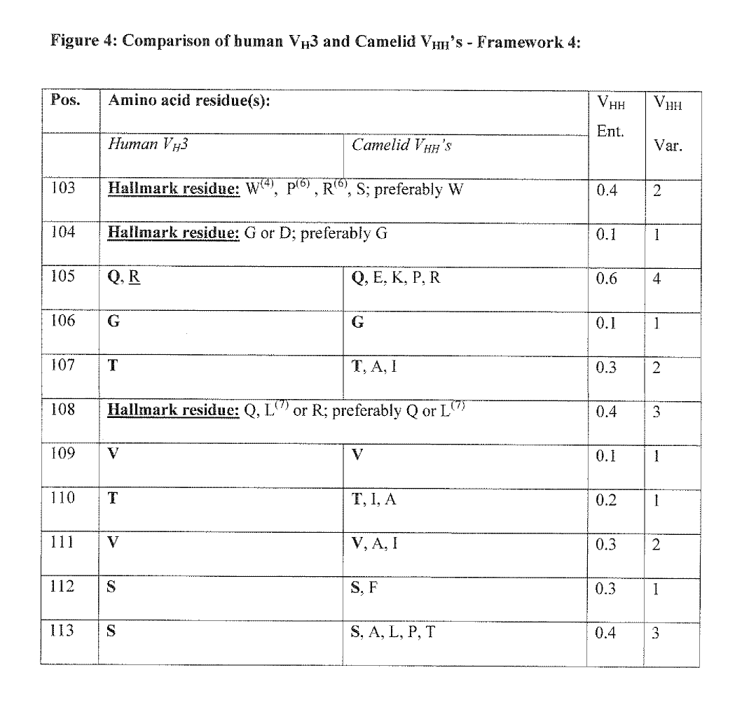

For example, WO 09/068,627, its sequence listing and its experimental part give specific examples of bivalent or multivalent, bispecific or multispecific, and/or biparatopic constructs that comprise 119A3 or one of its variants (see for example SEQ ID NO's: 2151 to 2159; 2533; 2537; 2539; 2541 to 2547; and 2615 to 2628; Examples 12 to 15, 24, 25 and 28-46; and FIGS. 4, 7, 8, 9, 42 and 45). It is envisaged that the amino acid sequences of the invention can replace 119A3 or its variants in such constructs, to provide or multivalent, bispecific or multispecific, and/or biparatopic constructs that are polypeptides of the (present) invention.

In one specific aspect, such a construct (i.e. polypeptide of the invention) is a biparatopic construct that comprises at least one amino acid sequence of the invention (and preferably only one) and at least one (and preferably only one) other Nanobody that can bind to the p19 subunit (for example, one of the Nanobodies described in WO 09/068,627). Such other Nanobody may for example be a p19+ sequence but is preferably a p19− sequence. In one specific aspect, such a biparatopic construct comprises an amino acid sequence of the invention and another Nanobody that is 81A12 (SEQ ID NO's: 1936 of WO 09/068,627), or a (humanized) variant thereof, such as one of 81A2v12 to 81A12v5 (SEQ ID NO's: 2580 to 2585 of WO 09/068,627) or an even further humanized variant thereof. Such a (humanized) variant of 81A12 may optionally also contain one or more substitutions essentially similar to one or more of the substitutions (a) to (c) above. In another specific aspect, such a biparatopic construct comprises an amino acid sequence of the invention and another Nanobody that is 81G2 (SEQ ID NO's: 1930 of WO 09/068,627), or a (humanized) variant thereof, such as one of 81G2v1 to 81G2v11 (SEQ ID NO's: 2586 to 2597 of WO 09/068,627) or an even further humanized variant thereof. Such a (humanized) variant of 81G2 may optionally also contain one or more substitutions essentially similar to one or more of the substitutions (a) to (c) above. Such constructs may again contain one or more further Nanobodies or other binding units, as well as suitable linkers and other functional groups, all as described in WO 09/068,627. For example, such constructs may be provided with increased half-life, for example through suitable modification such as through pegylation, by fusion to albumin, by including a Nanobody that can bind to serum albumin (such as the Nanobodies Alb-1 or Alb-8 described in WO 09/068,627, or one of the other serum-albumin binding Nanobodies described in WO 08/028,977), or by attachment of a serum albumin binding peptide, such as those described in WO 08/068,280, WO 09/127,691 or further improved variants of such peptides).

Thus, one preferred polypeptide of the invention essentially consists of an amino acid sequence of the invention (such as, preferably, 119A3v18, 119A3v20, 119A3v21 or 119A3v22) and a humanized variant of 81G2 (such as 81G2v11). To increase its half-life, such a polypeptide may be suitably pegylated, may be suitably fused to human serum albumin, or may comprise a Nanobody that can bind to human serum albumin (such as, preferably, Alb-8); all essentially as described in WO 08/028,977. The Nanobodies present in such a polypeptide of the invention may be suitably linked to each other, optionally via one or more suitable linkers, again essentially as described in WO 08/028,977. Some preferred, but non-limiting examples of such polypeptides of the invention are given in the sequence listing; and these polypeptides form preferred aspects of the invention.

Another preferred polypeptide of the invention essentially consists of an amino acid sequence of the invention (such as, preferably, 119A3v18, 119A3v20, 119A3v21 or 119A3v22) and a humanized variant of 81A12 (such as 81A12v5). To increase its half-life, such a polypeptide may be suitably pegylated, may be suitably fused to human serum albumin, or may comprise a Nanobody that can bind to human serum albumin (such as, preferably, Alb-8); all essentially as described in WO 08/028,977. The Nanobodies present in such a polypeptide of the invention may be suitably linked to each other, optionally via one or more suitable linkers, again essentially as described in WO 08/028,977. Some preferred, but non-limiting examples of such polypeptides of the invention are given in the sequence listing; and these polypeptides form preferred aspects of the invention.

Some (other) particularly preferred “polypeptides of the invention” as described herein may be essentially as described for the biparatopic constructs comprising 119A3 or a variant thereof in the U.S. provisional application 61/181,384 filed by Ablynx N. V. on May 27, 2009 and entitled “Biparatopic protein constructs directed against IL-23”, but comprising an amino acid sequence of the invention where such a biparatopic construct according to U.S. 61/181,384 comprises 119A3 or one of the variants thereof described in WO 09/068,627. In other words, some particularly preferred examples of polypeptides of the invention may be obtained simply by taking one of the biparatopic constructs as described in U.S. 61/181,384 that comprises 119A3 or a variant thereof, and replacing 119A3 or said variant with an amino acid sequence of the invention (for example, and preferably, with 119A3v18, 119A3v20, 119A3v21 or 119A3v22).

In one specific, but non-limiting aspect, a biparatopic protein or polypeptide of the present invention may comprise one binding domain that is a variant or analog of 119A3 (and in particular a humanized variant 119A3, which may for example be as further described herein) and one binding domain which is variant or analog of 81A12 (and in particular a humanized variant 81A12, which may for example be as further described herein), in which the binding domain that is a variant or analog of 81A12 (and in particular a humanized variant 81A12) is towards the N-terminus (i.e. “upstream of”) of the protein or polypeptide compared to the binding domain that is a variant or analog of 119A3 (and in particular a humanized variant 119A3, which may for example be as further described herein). Such biparatopic constructs with the 81A12-based binding unit towards the N-terminus may further essentially be as described in PCT/EP2008/066365; and may for example contain one or more further Nanobodies or other binding units, as well as suitable linkers and other functional groups, all as described in WO 09/068,627. For example, such constructs may be provided with increased half-life, for example through suitable modification such as through pegylation, by fusion to albumin, by including a Nanobody that can bind to serum albumin (such as the Nanobodies Alb-1 or Alb-8 described in WO 09/068,627, or one of the other serum-albumin binding Nanobodies described in WO 08/028,977), or by attachment of a serum albumin binding peptide, such as those described in WO 08/068,280, WO 09/127,691 or further improved variants of such peptides).

Some non-limiting examples of such proteins and polypeptides with the 81A12-based binding unit towards the N-terminus may be represented as follows (with the N-terminus of the polypeptide towards the right and the C-terminus towards the left):
[81A12-based binding domain]-linker-[119A3-based binding domain], which construct may optionally be pegylated for increased half-life;
[81A12-based binding domain]-linker-[Nanobody binding to serum albumin, such as Alb-1 or Alb-8]-linker-[119A3-based binding domain];
[serum albumin]-linker-[81A12-based binding domain]-linker-[119A3-based binding domain];
[81A12-based binding domain]-linker-[119A3-based binding domain]-linker-[serum albumin]
[serum albumin binding peptide (monovalent or in tandem)]-[81A12-based binding domain]-linker-[119A3-based binding domain];
[81A12-based binding domain]-linker-[119A3-based binding domain]-[serum albumin binding peptide (monovalent or in tandem)].

It may be that polypeptides of the invention in which the 81A12-based binding domain is located towards the N-terminus (i.e. relative to the 119A3-based binding domain) may have one or more favourable properties compared to the corresponding constructs in which the 119A3-based binding domain is located towards the N-terminus (i.e. relative to the 81A12-based binding domain). For example, polypeptides in which the 81A12-based binding domain is located towards the N-terminus may show higher expression or production yields compared to corresponding construct in which the 119A3-based binding domain is located towards the N-terminus. Reference is made to the Experimental Part.

Constructs with the 119A3-based binding unit towards the N-terminus may for example be formatted as follows:
[119A3-based binding domain]-linker-[81A12-based binding domain], which construct may optionally be pegylated for increased half-life;
[119A3-based binding domain]-linker-[Nanobody binding to serum albumin, such as Alb-1 or Alb-8]-linker-[81A12-based binding domain];
[serum albumin]-linker-[119A3-based binding domain]-linker-[81A12-based binding domain];
[119A3-based binding domain]-linker-[81A12-based binding domain]-linker-[serum albumin]
[serum albumin binding peptide (monovalent or in tandem)]-[119A3-based binding domain]-linker-[81A12-based binding domain];
[119A3-based binding domain]-linker-[81A12-based binding domain]-[serum albumin binding peptide (monovalent or in tandem)].

Preferably, all amino acid sequences of the invention and polypeptides of the invention are capable of undergoing essentially the same binding interactions as described in the U.S. provisional application 61/181,384 for 119A3, variants of 119A3 and polypeptides comprising 119A3 or variants thereof. Reference is for example made to Example 1.

The polypeptides of the invention preferably have a melting point (Tm) determined using DSC (under the conditions set out in the Experimental Part) of more than 60° C.

Also, amino acid sequences of the invention preferably have an affinity for p19 (as measured using the BIACORE assay described in Example 12 of WO 09/068,627) that is essentially the same or better than the affinity of 119A3 for p19, and more preferably an affinity for p19 that is essentially the same or better than the affinity of 119A3v17 for p19.

Furthermore, amino acid sequences of the invention preferably have a potency (as measured using the alpha-screen assay described in Example 22 of WO 09/068,627) that is essentially the same or better than the potency of 119A3, and more preferably a potency that is essentially the same or better than potency of 119A3v17.

Moreover, amino acid sequences of the invention preferably have a neutralizing activity in a mouse splenocyte assay (see Examples 15 and 23 of WO 09/068,627) that is essentially the same or better than the neutralizing activity of 119A3, and more preferably a neutralizing activity that is essentially the same or better than neutralizing activity of 119A3v17.

Moreover, amino acid sequences of the invention preferably have a neutralizing activity in a mouse splenocyte assay (see Examples 15 and 23 of WO 09/068,627) that is essentially the same or better than the neutralizing activity of 119A3, and more preferably a neutralizing activity that is essentially the same or better than neutralizing activity of 119A3v17.

Polypeptides of the invention preferably have a neutralizing activity (expressed as IC50) in a mouse splenocyte assay using hIL-23 (see Example 30 of WO 09/068,627) that is better than (i.e. less than) 50 μM, preferably better than 20 μM, more preferably better than 10 μM such as between 8 and 1 μM or less.

Possible applications and uses of the amino acid sequences and polypeptides of the invention (and of compositions comprising the same) are mentioned throughout WO 09/068,627 (see for example pages 7/8, 32 and 328 to 337 of WO 09/068,627). Other aspects, embodiments, applications and uses of such constructs are described throughout the disclosure of WO 09/068,627 (see for example the reference to p19+ sequences on pages 49-51), and such bivalent or multivalent, bispecific or multispecific, and/or biparatopic constructs that comprise, next to one or more amino acid sequences of the invention form a further aspect of the invention.

Generally, these may include use in (pharmaceutical composition for) the prevention and/or treatment of diseases and disorders associated with heterodimeric cytokines and their receptors (and in particular, with IL-23 or IL-23 mediated signaling), which as mentioned in WO 09/068,627 are diseases and disorders that can be prevented and/or treated, respectively, by suitably administering to a subject in need thereof (i.e. having the disease or disorder or at least one symptom thereof and/or at risk of attracting or developing the disease or disorder) of either a polypeptide or composition of the invention (and in particular, of a pharmaceutically active amount thereof) and/or of a known active principle active against heterodimeric cytokines (and in particular, IL-23) and/or their receptors or a biological pathway or mechanism in which heterodimeric cytokines (and in particular, IL-23) and/or their receptors is involved (and in particular, of a pharmaceutically active amount thereof). Examples of such diseases and disorders associated with heterodimeric cytokines and their receptors will be clear to the skilled person based on the disclosure herein, and for example include the following diseases and disorders: inflammation and inflammatory disorders such as bowel diseases (colitis, Crohn'disease, IBD), infectious diseases, psioriasis, cancer, autoimmune diseases (such as MS), carcoidis, transplant rejection, cystic fibrosis, asthma, chronic obstructive pulmonary disease, rheumatoid arthritis, viral infection, common variable immunodeficiency, and the various diseases and disorders mentioned in the prior art cited herein. Based thereon, it will also be clear to the skilled person with heterodimeric cytokines (and/or receptors thereof) are involved in which specific diseases and disorders.

For example, as mentioned on pages 4-5 of WO 09/068,627, IL23 was shown to be responsible for the chronic inflammation observed in inflammatory bowel disease. This was confirmed by the fact that the IL23R gene was identified as being involved in inflammatory bowel disease. It has also been found that p19 knock out mice are resistant to collagen-induced arthritis and colitis, whereas comparable p35 knock out mice were found to be more susceptible to collagen-induced arthritis. Also, when p19 knock out mice were crossed with IL-10 knock out mice, the resulting offspring were resistant to colitis, whereas similar crosses of p19 knock out mice with IL-10 knock out mice resulted in offspring that was susceptible to colitis. It was further found that a monoclonal antibody against p19 inhibits the development of EAE, a preclinical animal model for multiple sclerosis, and reduces serum levels of IL-17 (which is not regulated by IL-12). Also, IL-23 rather than IL-12 appears to be the essential cytokine in CNS autoimmune inflammation. All this results suggests that IL-23/p19 may be an attractive target for the treatment of colitis, Crohn's diseases, IBD, multiple sclerosis, rheumatoid arthritis and some of the other diseases and disorders mentioned herein. Also, IL23 and IL27—two of the other heterodimeric cytokines from the IL-12 family—also regulate TH1-cell response, albeit with distinct functions. The ability of IL-23 to stimulate CD4+ T cells to produce IL-17 also has been described as having a dominant role in the development and maintenance of autoimmune inflammation.

Also, Example 45 of WO 09/068,627 shows that the polypeptides of WO 09/068,627 (and thus, by extension, the polypeptides of the invention) can also be valuable in the prevention and treatment of psoriasis (either by systemic/parenteral administration or by topical treatment, e.g. using a creme or lotion (see page 328 and 331-332 of WO 09/068,627).

In another aspect, the invention relates to a nucleic acid that encodes an amino acid sequence of the invention or a polypeptide of the invention (or a suitable fragment thereof). Such a nucleic acid will also be referred to herein as a “nucleic acid of the invention” and may for example be as further described in WO 09/068,627; and may in particular be in the form of a genetic construct, again as further described in WO 09/068,627 (see for example pages 316-320).

In another aspect, the invention relates to a host or host cell that expresses (or that under suitable circumstances is capable of expressing) an amino acid sequence of the invention) and/or a polypeptide of the invention; and/or that contains a nucleic acid of the invention. Such a host or host cell may again generally be as described in WO 09/068,627 (see for example pages 315-328).

The invention also relates to methods for the production/expression of the amino acid sequences and polypeptides of the invention. Such methods may generally comprise the steps of (i) the expression, in a suitable host cell or host organism or in another suitable expression system of a nucleic acid that encodes an amino acid sequence or polypeptide of the invention, optionally followed by: (ii) isolating and/or purifying the amino acid sequence or polypeptide of the invention thus obtained. In particular, such a method may comprise the steps of (i) cultivating and/or maintaining a host of the invention under conditions that are such that said host of the invention expresses and/or produces at least one amino acid sequence and/or polypeptide of the invention; optionally followed by (ii) isolating and/or purifying the amino acid sequence, Nanobody or polypeptide of the invention thus obtained. These methods again may essentially be performed as described in WO 09/068,627 (see for example pages 315-328).

One specific method for the production/expression of the amino acid sequences and polypeptides of the invention is described in the International application of Ablynx N. V. entitled “Method for the production of domain antibodies”, which has an international filing date of Apr. 30, 2010.

The invention further relates to a product or composition containing or comprising at least one amino acid sequence of the invention, at least one polypeptide of the invention (or a suitable fragment thereof) and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Such products or compositions may again generally be as described in WO 09/068,627 (see for example pages 329-337).

The invention also relates to the use of an amino acid sequence or polypeptide of the invention, or of a composition comprising the same, in (methods or compositions for) modulating (as defined in WO 09/068,627) IL-23 and/or IL-23-mediated signalling (as defined in WO 09/068,627), either in vitro (e.g. in an in vitro or cellular assay) or in vivo (e.g. in an a single cell or in a multicellular organism, and in particular in a mammal, and more in particular in a human being, such as in a human being that is at risk of or suffers from a disease or disorder associated with heterodimeric cytokines and their receptors).

The invention also relates to methods for modulating (as defined in WO 09/068,627) IL-23 and/or IL-23-mediated signalling (as defined in WO 09/068,627), either in vitro (e.g. in an in vitro or cellular assay) or in vivo (e.g. in an a single cell or multicellular organism, and in particular in a mammal, and more in particular in a human being, such as in a human being that is at risk of or suffers from a disease or disorder associated with IL-23 and/or its receptors), which method comprises at least the step of contacting IL-23 with at least one amino acid sequence or polypeptide of the invention, or with a composition comprising the same, in a manner and in an amount suitable to modulate IL-23 and/or IL-23-mediated signalling.

The invention also relates to the use of an one amino acid sequence or polypeptide of the invention in the preparation of a composition (such as, without limitation, a pharmaceutical composition or preparation as further described herein) for modulating (as defined in WO 09/068,627) IL-23 and/or IL-23-mediated signalling (as defined in WO 09/068,627), either in vitro (e.g. in an in vitro or cellular assay) or in vivo (e.g. in an a single cell or multicellular organism, and in particular in a mammal, and more in particular in a human being, such as in a human being that is at risk of or suffers from a disease or disorder associated with heterodimeric cytokines and their receptors).

The invention will now be further described by reference to the following non-limiting Experimental Part and the attached non-limiting sequence listing and the non-limiting Figures, in which:

FIGS. 1-4 are tables giving sequence comparisons of the framework regions of human V_{H}3 domains and V_{HH} sequences (data taken from WO 09/068,627).

FIG. 5 gives an alignment of 119A3 (WO 09/068,627), 119A3v16 (WO 09/068,627) and 119A3v18 (invention).

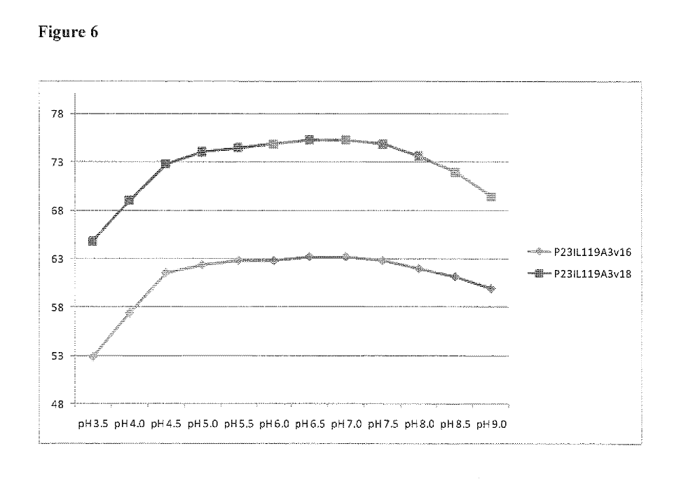

FIG. 6 gives the melting temperatures (y-axis, Tm in ° C.) of two humanized variants of 119A3 (119A3v16—WO 09/068,627; and 119A3v18 invention) as determined by TSA at different pH values (x-axis).

### EXPERIMENTAL PART

Based upon the crystal structure and molecular modeling, it was hypothesized that the physical stability of the humanized variant 119A3v16 (see WO 09/068,627, SEQ ID NO: 2578) could be improved by a mutation L78V.

The increased stability after back mutation of L78V was experimentally confirmed: a small amount of His-myc tagged 119A3v18 (containing the mutation L78V) was prepared and the melting temperature was determined by thermal shift assay (TSA) in comparison with the 119A3v16. FIG. 6 clearly shows that indeed a significant increase in the melting temperature of 12° C. could be observed thus confirming that the L78V mutation in the core of the protein improved the stability of this Nanobody®. The relative stability (Tm) in the pH range tested was comparable.

Melting curves were also obtained using Differential Scanning calorimetry for biparatopic constructs based on the 119A3v18 building block and the 81A12 building block (again, compared to similar constructs based on the 119A3v16 building block), and these confirmed the clear improvement in thermal stability that the mutation L78V in 119A3 provides, also when the building blocks are incorporated in a biparatopic format (data not shown). The data also show that tested constructs with the 81A12-based building block towards the N-terminus (SEQ ID NO:s 35 and 38) have higher melting temperatures than the tested constructs with the 119A3-based building block towards the N-terminus.

To investigate the influence of other substitutions on the properties of the amino acid sequences of the invention, peptide map analysis of 119A3v18 after chemical stress was performed (again, compared to 119A3v16).

For example, the reference biparatopic construct 119A3v16-9GS-A1b8-90S-81A12v5 (which is based on the building blocks 119A3v16 and 81A12v5 as described in WO 09/068,627) was found to show a certain degree of de-amidation of the N52 (Kabat N°), located in the CDR2 of 119A3v16 building block. This variant was resolved in the cIEF analysis of samples formulated at 25 mg/mL in a L-Histidine buffer pH6, with 0.05% (v:v) Tween 80 and 10% (w:v) sucrose, after storage during 6 weeks at 37 and 25° C., and was confirmed by analysis of forced deamidation samples (storage in ammonium carbonate buffer at pH9; during 3 days at RT) by LC/MS peptide map analysis.

Thus, further variants of 119A3v18 (invention) called 119A3v20, 119A3v21 and 119A3v22 were designed that comprise, compared to 119A3v18, one or both of the substitutions N52E or S82bN. Peptide maps of these variants after chemical stress (3 days at pH 9 at RT) were determined (data not shown) and this showed that N52 may be more was sensitive to de-amidation than N82a.

Surprisingly, the comparison of the peptide maps of 119A3v16 (WO 09/068,627) and 119A3v18 (invention) showed that the rate of de-amidation of N52 is decreased in 119A3v18 demonstrating that the increased thermodynamic stability achieved by the L78V mutation (without the further N52E or S82bN mutations) also already resulted in an increased chemical stability of the molecule.

The potency of biparatopic constructs comprising an amino acid sequence of the invention was compared to the potency of a comparable biparatopic construct comprising the 119A3v16 building block according to WO 09/068,627. The constructs tested were those of SEQ ID NO's: 35, 36, 37 and 38. These were compared to the reference construct 119A3v16-9GS-Alb8-9GS-81A12v5 (which is based on the building blocks 119A3v16 and 81A 2v5 as described in WO 09/068,627). The assay used was the mouse splenocyte assay essentially as described in Examples 15 and 25 of WO 09/068,627. When, in a first experiment, the constructs of SEQ ID NO's: 35, 36 and 37 were compared to the reference, they showed comparable potency (1050 in nm of 0.033, 0.028 and 0.032 for SEQ ID NO's: 35, 36 and 37, respectively, compared to 0.028 for the reference construct). In a second experiment, the construct of SEQ ID NO: 38 showed an 1050 of 0.039, compared to 0.030 for the reference construct. This shows that the L78V substitution in the amino acid sequences of the invention (and in constructs comprising the same) has no major influence on potency, compared to the 1193b16 building block described in WO 09/068,627).

To determine the influence of the mutation L78V on expression levels, expression levels of the constructs of SEQ ID NO's 35, 36, 37 and 38 were compared to the expression levels of the reference construct 119A3v16-9GS-A1b8-9GS-81A12v5, using a generic high-cell density fermentation process in the Pichia pastoris strain X-33 (Invitrogen). The Ably1 medium, a rich medium containing tryptone as complex component, and standard fermentation parameters such as 30° C., pH5 and 30% dissolved oxygen were used. After the batch phase, a glycerol fed-batch was applied until a Wet Cell Weight of approximately 400 g/L was achieved. Hereafter, induction was started by adding MeOH to the culture. To adapt the culture to MeOH as C-source, an adaptation phase was performed (2 hrs 1.5 mL/L·h followed by 2 hrs at 3 mL/L·h) followed by a constant feed rate of 4 ml/h/L until the end of fermentation (114 hrs total induction time). The fermentation samples were analyzed by RPC analysis, after a proteinA sample clean up, to check for product related variants. Briefly: clarified culture supernatant is mixed with a fixed amount of ProtA resin, and eluted in MQ containing TEA 0.1% and as such are ready for loading on RPC. The results are shown in the Table below, which shows that the increased Tm in DSC of the amino acids sequences/polypeptides of the invention corresponds to a marked increase in expression yields.

TABLE
Estimation of the purity and the yield of intact protein determined
via RPC analysis after protein A clean-up and Cu2+ treatment
of the cell free medium.

| | Tm | Total conc. in cell | conc. |
|---|---|---|---|
| | DSC | free medium | intact material |
| Construct | ° C. | g/L* | g/L* |
| Reference | 59.0 | 0.5 | 0.4 |
| SEQ ID NO: 35 | 63.8 | 1.2 | 0.8** |
| SEQ ID NO: 38 | 64.1 | 1.2 | 1.1 |
| SEQ ID NO: 36 | 62.0 | 1.4 | 1.2 |
| SEQ ID NO: 37 | 63.3 | 1.2 | 0.4** |

*Based on RPC results after Cu2+ treatment for complete formation of all disulfide bridges and protein A clean-up.
**The major impurities present in these samples are protein with a part of miss cleaved signal sequence attached to the N-terminus, which can be avoided by adapting the fermentation conditions.

1. Amino acid sequence that is a variant of PMP119A3 (SEQ ID NO:1) that comprises, compared to the amino acid sequence of PMP119A3, (i) at least one and preferably both of the mutations H37Y and M43K; (ii) a valine residue at position 78; (iii) at least one, preferably at least two, and more preferably three, four of five humanizing substitutions; (iv) as well as optionally one or more further suitable amino acid substitutions.

2. Amino acid sequence that is a variant of 119A3(H37Y-M43K) (SEQ ID NO:2) that comprises, compared to the amino acid sequence of PMP119A3(H37Y-M43K), (i) a valine residue at position 78; (ii) at least one, preferably at least two, and more preferably three, four of five humanizing substitutions; (iii) as well as optionally one or more further suitable amino acid substitutions.

3. Amino acid sequence that is a variant of 119A3v17 (SEQ ID NO:3) that comprises, compared to the amino acid sequence of 119A3v17, (i) a valine residue at position 78; (ii) optionally 1 to 5, such as one, two or three further amino acid differences compared to the sequence of 119A3v17.

4. Amino acid sequence, chosen from 119A3v18(SEQ ID NO:4), 119A3v20 (SEQ ID NO:5), 119A3v21 (SEQ ID NO:6) or 119A3v22 (SEQ ID NO: 7).

5. Protein or polypeptide that essentially consists of an amino acid sequence according to claim 1.

6. Protein or polypeptide that comprises an amino acid sequence according to claim 1.

7. Protein or polypeptide that comprises an amino acid sequence according to claim 1 and one or more other groups, residues, moieties, binding domains or binding units.

8. Protein or polypeptide that comprises an amino acid sequence according to claim 1 and one or more other immunoglobulin single variable domains, VHH's, (single) domain antibodies, Nanobodies or dAb's.

9. Protein or polypeptide that comprises an amino acid sequence according to claim 1 and one or more other immunoglobulin single variable domains, VHH's, (single) domain antibodies, Nanobodies or dAb's against the p19 subunit of IL-23.

10. Protein or polypeptide that comprises an amino acid sequence according to claim 1 and one or more other immunoglobulin single variable domains, VHH's, (single) domain antibodies, Nanobodies or dAb's against the p40 subunit of IL-23.

11. Protein or polypeptide that comprises an amino acid sequence according to claim 1 and another Nanobody that is a Nanobody against the p40 subunit of IL-23.

12. Protein or polypeptide that comprises an amino acid sequence according to claim 1 and another Nanobody that is a Nanobody against the p19 subunit of IL-23.

13. Protein or polypeptide that comprises an amino acid sequence according to claim 1 and another Nanobody that is a Nanobody against the p19 subunit of IL-23.

14. Protein or polypeptide that comprises an amino acid sequence according to claim 1 and another Nanobody that is 81A12 (SEQ ID NOs: 1936 of WO 09/068,627), or a (humanized) variant thereof, such as one of 81A2v12 to 81A12v5 (SEQ ID NOs: 2580 to 2585 of WO 09/068,627) or an even further humanized variant thereof.

15. Protein or polypeptide that comprises an amino acid sequence according to claim 1 and another Nanobody that is 81G2 (SEQ ID NOs: 1930 of WO 09/068,627), or a (humanized) variant thereof, such as one of 81G2v1 to 81G2v11 (SEQ ID NOs: 2586 to 2597 of WO 09/068,627) or an even further humanized variant thereof.

16. Protein or polypeptide according to claim 7 that has been provided with increased half-life, for example through suitable modification such as through pegylation, by fusion to albumin, by including a Nanobody that can bind to serum albumin, or by attachment of a serum albumin binding peptide.


SEQUENCE LISTING (40 sequences)

SEQ ID NO: 1 - protein, 125 aa, Artificial Sequence
  Nanobody or Nanobody construct
EVQLVESGGGLVQAGGSLRLSCAASGRIFSLPASGNIFNLLTIAWHRQAPGMQRELVATINSGSRTNYADSVKGRFTISRDNAQKTVYLQMNNLKPEDTAVYYCQTSGSGSPNFWGQGTQVTVSS

SEQ ID NO: 2 - protein, 125 aa, Artificial Sequence
  Nanobody or Nanobody construct
EVQLVESGGGLVQAGGSLRLSCAASGRIFSLPASGNIFNLLTIAWYRQAPGKQRELVATINSGSRTNYADSVKGRFTISRDNAQKTVYLQMNNLKPEDTAVYYCQTSGSGSPNFWGQGTQVTVSS

SEQ ID NO: 3 - protein, 125 aa, Artificial Sequence
  Nanobody or Nanobody construct
EVQLLESGGGLVQPGGSLRLSCAASGRIFSLPASGNIFNLLTIAWYRQAPGKGRELVSTINSGSRTYYADSVKGRFTISRDNSKKTLYLQMNSLRPEDTAVYYCQTSGSGSPNFWGQGTLVTVSS

SEQ ID NO: 4 - protein, 125 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 5 - protein, 125 aa, Artificial Sequence
  Nanobody or Nanobody construct
EVQLLESGGGLVQPGGSLRLSCAASGRIFSLPASGNIFNLLTIAWYRQAPGKGRELVATIESGSRTYYADSVKGRFTISRDNSKKTVYLQMNSLRPEDTAVYYCQTSGSGSPNFWGQGTLVTVSS

SEQ ID NO: 6 - protein, 125 aa, Artificial Sequence
  Nanobody or Nanobody construct
EVQLLESGGGLVQPGGSLRLSCAASGRIFSLPASGNIFNLLTIAWYRQAPGKGRELVATIESGSRTYYADSVKGRFTISRDNSKKTVYLQMNNLRPEDTAVYYCQTSGSGSPNFWGQGTLVTVSS

SEQ ID NO: 7 - protein, 125 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNNLRPEDTAVYYCQTSGSGSPNFWGQGTLVTVSS

SEQ ID NO: 8 - protein, 386 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGRTLSSYAMGWFRQAPGKGREFVSRISQGGTAIYYADSVKGRFTISRDNSKNTLYLQMNSLRPEDTAVYYCAKDPSPYYRGSAYLLSGSYDSWGQGTLVTVSS

SEQ ID NO: 9 - protein, 386 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLLESGGGLVQPGGSLRLSCAASGRTLSSYAMGWFRQAPGKGREFVSRISQGGTAIYYADSVKGRFTISRDNSKNTLYLQMNSLRPEDTAVYYCAKDPSPYYRGSAYLLSGSYDSWGQGTLVTVSS

SEQ ID NO: 10 - protein, 292 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLLESGGGLVQPGGSLRLSCAASGLPFSTKSMGWFRQAPGKGREFVSRISPGGTSRYYGDFVKGRFTISRDNSKNTLYLQMNSLRAEDTAVYYCASGERSTYIGSNYYRTNEYDYWGQGTLVTVSSGGGC

SEQ ID NO: 11 - protein, 873 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLLESGGGLVQPGGSLRLSCAASGLPFSTKSMGWFRQAPGKGREFVSRISPGGTSRYYGDFVKGRFTISRDNSKNTLYLQMNSLRAEDTAVYYCASGERSTYIGSNYYRTNEYDYWGQGTLVTVSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 12 - protein, 386 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGRTLSSYAMGWFRQAPGKGREFVARISQGGTAIYYADSVKGRFTISRDNSKNTLYLQMNSLRPEDTAVYYCAKDPSPYYRGSAYLLSGSYDSWGQGTLVTVSS

SEQ ID NO: 13 - protein, 287 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQAGGSLRLSCAAPGRTFSEYAMGWFRQNPGNEREFVAAISRGGGFTDYADSVKGRFTISRDNAKNTVYLQMNSLKPEDTAVYYCAADGRSSFGIAKRSRYEYDSWGQGTQVTVSS

SEQ ID NO: 14 - protein, 290 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQAGGSLTLSCAASVRTFSTSAMGWFRQAPGKEREFVAAINWNAGSTYHADSVKGRFTISGDNAKNSVFLQMNGLKPEDTAVYYCAAQASGRVTYFDYAYRRGSNYDYWGQGTQVTVSS

SEQ ID NO: 15 - protein, 289 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQAGGSLTLSCAVSGLTFNNYAMGWFRQVPGKEREFVAAISRSGLRTFYSDSVKGRFTISRDSAKSTVYVQMNNLKPEDTAVYFCAADRRTYYSEGPSLTDTLRYDYWGQGTQVTVSS

SEQ ID NO: 16 - protein, 386 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGLVQPGGSLRLSCIASGLPFSTKSMGWFRQAPGKEREFVARISPGGTSRYYGDFVKGRFAISRDNAKNTTWLQMNSLKAEDTAVYYCASGERSTYIGSNYYRTNEYDYWGTGTQVTVSS

SEQ ID NO: 17 - protein, 386 aa, Artificial Sequence; identical to SEQ ID NO: 16
  Nanobody or Nanobody construct

SEQ ID NO: 18 - protein, 292 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQPGGSLRLSCIASGLPFSTKSMGWFRQAPGKEREFVARISPGGTSRYYGDFVKGRFAISRDNAKNTTWLQMNSLKAEDTAVYYCASGERSTYIGSNYYRTNEYDYWGTGTQVTVSSGGGC

SEQ ID NO: 19 - protein, 873 aa, Artificial Sequence [sequence omitted]

SEQ ID NO: 20 - protein, 412 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQPGGSLRLSCIASGLPFSTKSMGWFRQAPGKEREFVARISPGGTSRYYGDFVKGRFAISRDNAKNTTWLQMNSLKAEDTAVYYCASGERSTYIGSNYYRTNEYDYWGTGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSS

SEQ ID NO: 21 - protein, 386 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGLVQAGGSLRLSCAASGRTLSSYAMGWFRQAPGKEREFVARISQGGTAIYYADSVKGRFTISRDNAKNTVYLQMNSLKPEDTAVYYCAKDPSPYYRGSAYLLSGSYDSWGQGTQVTVSS

SEQ ID NO: 22 - protein, 288 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQAGGSLRLSCAASGRTSSSYSMGWFRQAPGKEREFVAYISGGGLTIYYAHSVKGRFTISRDNAKNTVYLQMNSLNSADTAVYSCAAPKRDLYLGSRYPVPESYDYWGQGTQVTVSS

SEQ ID NO: 23 - protein, 285 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQPGGSLRLSCAASGFYLDDYAIAWFRQAPGKEREGVSGIDSGDGSTYYADSVKGRFTISSDNAKNTVYLQMNSLRPEDTAVYYCARVRTGWGLNAPDYAMDYWGKGTLVTVSS

SEQ ID NO: 24 - protein, 284 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQPGGSLRLSCAASGFTFSNYWMYWVRQAPGKGLERVAPLNPGGTITLYADFVKSRFTISRDNAKNTVYLQMNSLKPEDTAVYVCAAPSGTLYTRNKSGYIYWGQGTQVTVSS

SEQ ID NO: 25 - protein, 288 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQAGGSLRLSCAASGRTLSSYAMGWFRQAPGKEREFVARISQGGTAIYYADSVKGRFTISRDNAKNTVYLQMNSLKPEDTAVYYCAKDPSPYYRGSAYLLSGSYDSWGQGTQVTVSS

SEQ ID NO: 26 - protein, 288 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQPGGSLRLSCIASGLPFSTKSMGWFRQAPGKEREFVARISPGGTSRYYGDFVKGRFAISRDNAKNTTWLQMNSLKAEDTAVYYCASGERSTYIGSNYYRTNEYDYWGTGTQVTVSS

SEQ ID NO: 27 - protein, 285 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQPGGSLRLSCAASGFTLDDYAIAWFRQAPGKEREGVSGIDSGDGSAYYADSVKGRFTISSDNAKNTVYLQMNSLRPEDTAVYYCARVRTGWGLNAPDYAMDYWGKGTLVTVSS

SEQ ID NO: 28 - protein, 378 aa, Artificial Sequence
  Nanobody or Nanobody construct
EVQLVESGGGLVQPGGSLRLSCAASGFTFSSSVMFWLRQAPGKGLEWLSTIKPTGSTIYPGSGKGRFTISRDNAKNTLYLQINNLKPEDTALYYCARDIGGTVRGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 29 - protein, 388 aa, Artificial Sequence
  Nanobody or Nanobody construct
EVQLVESGGGSVQAGGSLRLSCKVSGIMFSDKGMGWFRQAPGKERETVAATNWSGGSTYYADSVKGRFTISRDNYKNTLHLQMASLKPEDTGIYYCAAAGDSWNSRRIEDYDYWGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 30 - protein, 386 aa, Artificial Sequence
  Nanobody or Nanobody construct
EVQLVESGGELVQAGGSLRLSCAASGSIVSISAMAWYRQAPGKQRERVATITRGGTPDYADSAKDRFTISRDNAKNTVSLQMNNLKPEDTAVYYCKAALRTPTHDYNYYDYWGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 31 - protein, 378 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGLVQPGGSLRLSCAASGFTFSSSVMFWLRQAPGKGLEWLSTIKPTGSTIYPGSGKGRFTISRDNAKNTLYLQINNLKPEDTALYYCARDIGGTVRGQGTQVTVSS

SEQ ID NO: 32 - protein, 388 aa, Artificial Sequence
  Nanobody or Nanobody construct
EVQLVESGGGLVQAGGSLRLSCAASGRIFSLPASGNIFNLLTIAWYRQAPGKQRELVATINSGSRTNYADSVKGRFTISRDNAQKTVYLQMNNLKPEDTAVYYCQTSGSGSPNFWGQGTQVTVSSGGGGSGGGGSGGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGSVQAGGSLRLSCKVSGIMFSDKGMGWFRQAPGKERETVAATNWSGGSTYYADSVKGRFTISRDNYKNTLHLQMASLKPEDTGIYYCAAAGDSWNSRRIEDYDYWGQGTQVTVSS

SEQ ID NO: 33 - protein, 386 aa, Artificial Sequence
  Nanobody or Nanobody construct
EVQLVESGGGLVQAGGSLRLSCAASGRIFSLPASGNIFNLLTIAWYRQAPGKQRELVATINSGSRTNYADSVKGRFTISRDNAQKTVYLQMNNLKPEDTAVYYCQTSGSGSPNFWGQGTQVTVSSGGGGSGGGGSGGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGELVQAGGSLRLSCAASGSIVSISAMAWYRQAPGKQRERVATITRGGTPDYADSAKDRFTISRDNAKNTVSLQMNNLKPEDTAVYYCKAALRTPTHDYNYYDYWGQGTQVTVSS

SEQ ID NO: 34 - protein, 398 aa, Artificial Sequence
  Nanobody or Nanobody construct
EVQLLESGGGLVQPGGSLRLSCAASGRTLSSYAMGWFRQAPGKGREFVARISQGGTAIYYADSVKGRFTISRDNSKNTLYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 35 - protein, 398 aa, Artificial Sequence
  Nanobody or Nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 36 - protein, 386 aa, Artificial Sequence; identical to SEQ ID NO: 12
  Nanobody or Nanobody construct

SEQ ID NO: 37 - protein, 386 aa, Artificial Sequence
  Nanobody or Nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSS

SEQ ID NO: 38 - protein, 398 aa, Artificial Sequence
  Nanobody or Nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATIESGSRTYYADSVKGRFTISRDNSKKTVYLQMNSLRPEDTAVYYCQTSGSGSPNFWGQGTLVTVSS

SEQ ID NO: 39 - protein, 398 aa, Artificial Sequence
  Nanobody or Nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATIESGSRTYYADSVKGRFTISRDNSKKTVYLQMNNLRPEDTAVYYCQTSGSGSPNFWGQGTLVTVSS

SEQ ID NO: 40 - protein, 398 aa, Artificial Sequence
  Nanobody or Nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNNLRPEDTAVYYCQTSGSGSPNFWGQGTLVTVSS